(19) United States
(12) Patent Application Publication
(10) Pub. No.: US 2024/0382154 A1
(43) Pub. Date: Nov. 21, 2024

(54) NECK-WORN MEDICAL DEVICE

(71) Applicant: SPINA CARE CORP, Houston, TX (US)

(72) Inventor: Ivailo DACHOV, Sofia (BG)

(73) Assignee: SPINA CARE CORP, Houston, TX (US)

(21) Appl. No.: 18/317,211

(22) Filed: May 15, 2023

(51) Int. Cl. A61B 5/00; A61B 5/0205

(52) U.S. Cl. CPC A61B 5/6822; A61B 5/0205; A61B 5/6843; A61B 5/02438; A61B 5/14552

(57) ABSTRACT

A neck-worn medical device having a main body having a superficial monitoring interface. The main body has two opposing arm portions connected to the main body by way of flexure elements. The arm portions provide biasing components that urge the connected securement members terminating in electrode nodes against the chest of the wearer. The neck-worn medical device is dimensioned and shaped to wrap around the neck of a subject in a flush engagement with the nape, whereby the biasing components and flexure elements enable a nominal contact force between the monitoring interface and the skin of the nape. The neck-worn medical device can measure or evaluated, continuously, in real-time, biomarkers or the like of a subject.

### BACKGROUND OF THE INVENTION

The present invention relates to physiological monitoring systems and, more particularly, to a neck-worn medical device configured to urge a physiological monitor with a nominal contact force against skin adjacent the cervical vertebrae and sternal portions of the wearer.

Noninvasive physiological monitoring, such as pulse oximetry, is vital to the healthcare of patients. Many noninvasive physiological monitoring systems, however, require that the device sensing the physiological parameters maintain an effective skin contact for an extended period. The cervical spine is one of the most suitable locations in the body for the collection and assessment of biological and physiological parameters; though, unfortunately, it is one of the hardest to reach places on one's body for self-assessment.

As can be seen, there is an unmet need for a neck-worn medical device configured to urge a physiological monitor with a nominal contact force against skin adjacent the cervical vertebrae and sternal portions of the wearer for physiological monitoring. This unmet need is at least in part addressed by the present invention.

### SUMMARY OF THE INVENTION

In one aspect of the present invention, a neck-worn medical device including the following: a monitoring interface having a main housing, the main housing terminating in opposing flexure elements; an arm portion operatively associated with each flexure element so that each arm portion is movable, relative the main housing, between a biased position and a plurality of actuated positions; a distal end of the arm portion housing a biasing component; and a securement member projecting from the biasing component until it terminates at a node for a chest sensor, wherein the biasing component is configured, with the arm portions in the biased position, to urge the node against a sternal portion of a wearer and urge a nominal contact force between the monitoring interface and a nape portion of the wearer.

These and other features, aspects and advantages of the present invention will become better understood with reference to the following drawings, description, and claims.

### BRIEF DESCRIPTION OF THE DRAWINGS

FIG. 1 is a front elevation view of an exemplary embodiment of the present invention.

FIG. 2 is a perspective view of an exemplary embodiment of the present invention.

FIG. 3 is a bottom perspective view of an exemplary embodiment of the present invention.

FIG. 4 is an exploded perspective view of an exemplary embodiment of the present invention.

FIG. 5 is a perspective view of an exemplary embodiment of a panel of the present invention.

FIG. 6 is a perspective view of an exemplary embodiment of a panel of the present invention.

FIG. 7 is a detailed perspective view of an exemplary embodiment of a flexure element 30 of the present invention.

FIG. 8 is a perspective view of an exemplary embodiment of a panel of the present invention.

FIG. 9 is a perspective view of an exemplary embodiment of the present invention.

FIG. 10 is a perspective view of an exemplary embodiment of a biasing component 50 of the present invention.

FIG. 11 is a perspective view of an exemplary embodiment of the present invention.

### DETAILED DESCRIPTION OF THE INVENTION

Throughout this disclosure, various scientific publications, patents and published patent specifications are referenced by an identifying citation. The disclosures of these publications, patents and published patent specifications are hereby incorporated by reference into the present disclosure to more fully describe the state of the art to which this disclosure pertains.

As used herein, certain terms may have the following defined meanings.

As used in the specification and the claims, the singular form “a,” “an” and “the” include singular and plural references unless the context clearly dictates otherwise. For example, the term “biomarker” includes a single or plurality of biomarkers selected from

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a neck-worn medical device having a main body providing a superficial monitoring interface. The main body has two opposing arm portions connected to the main body by way of flexure elements. The arm portions provide biasing components that urge connected securement members terminating in electrode nodes against the chest of the wearer. The neck-worn medical device is dimensioned and adapted to wrap around the neck of a subject in a flush engagement with the nape, whereby the biasing components and flexure elements enable nominal contact force between the monitoring interface and the skin of the nape.

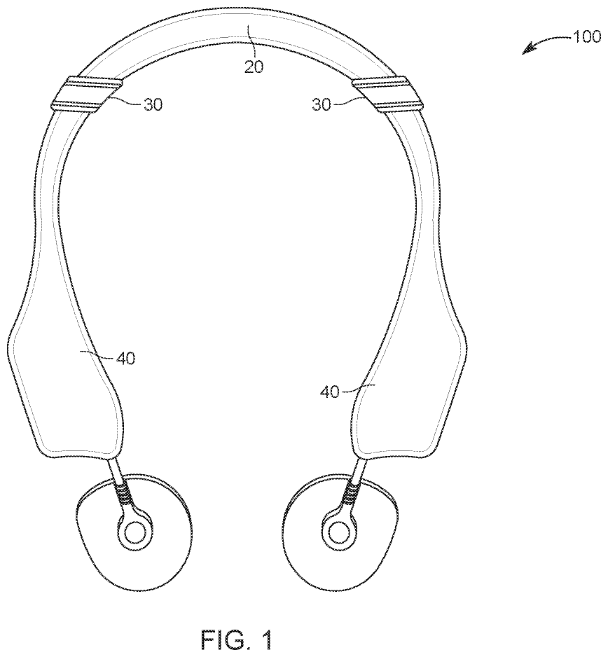

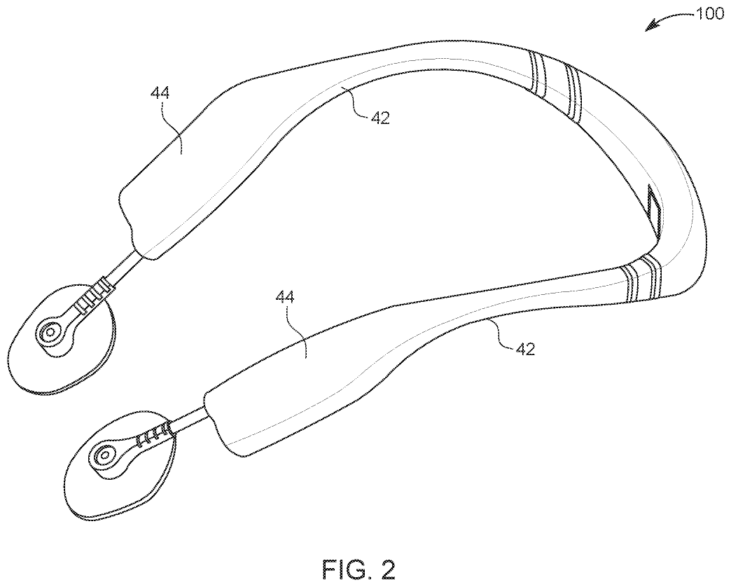

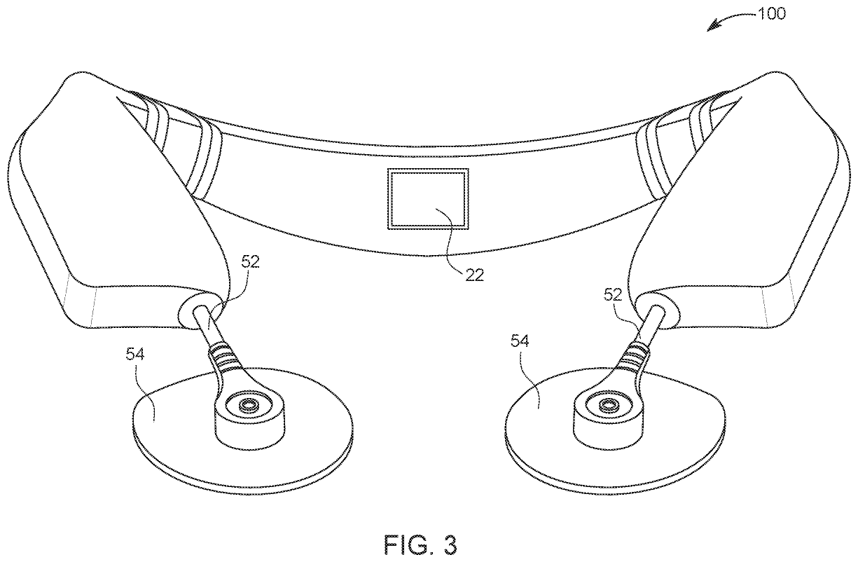

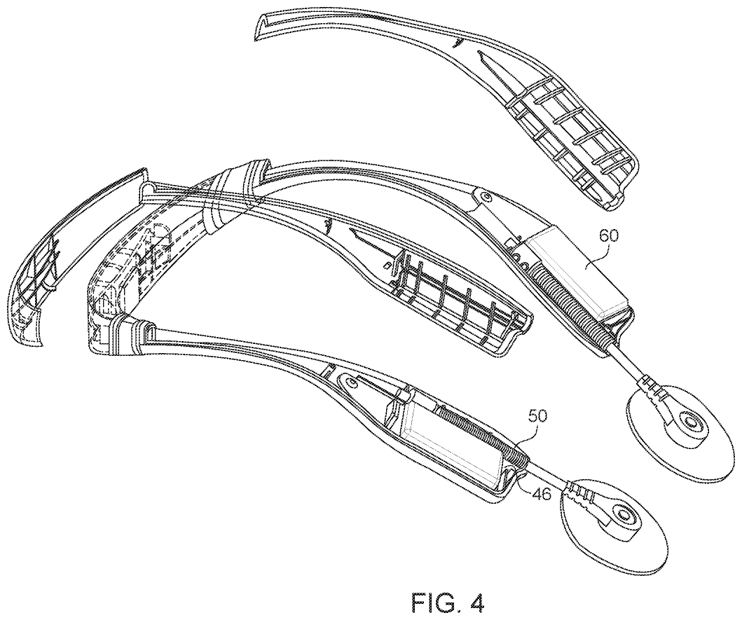

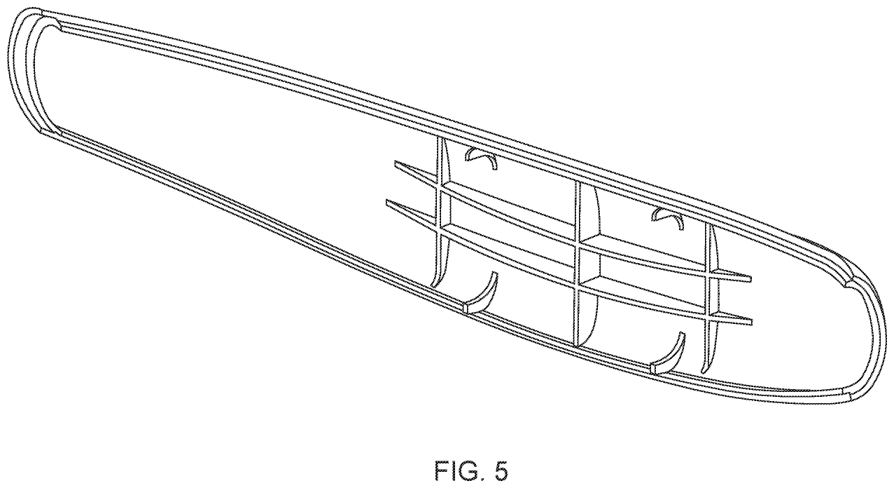

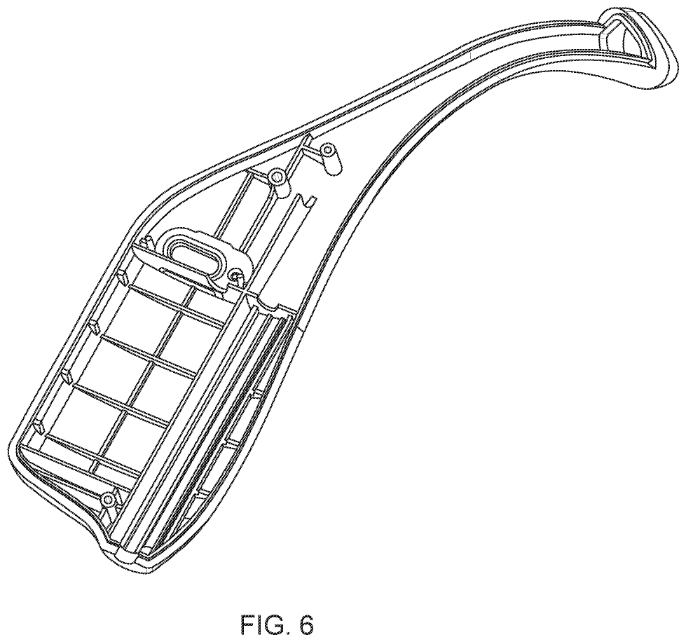

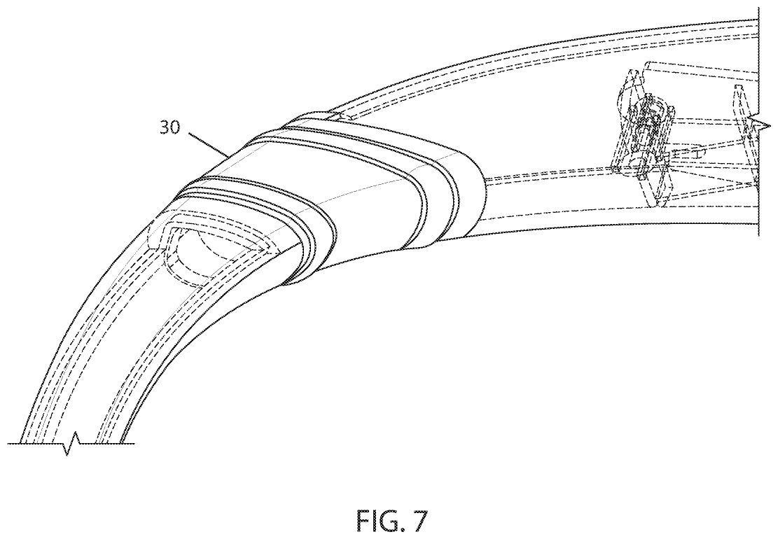

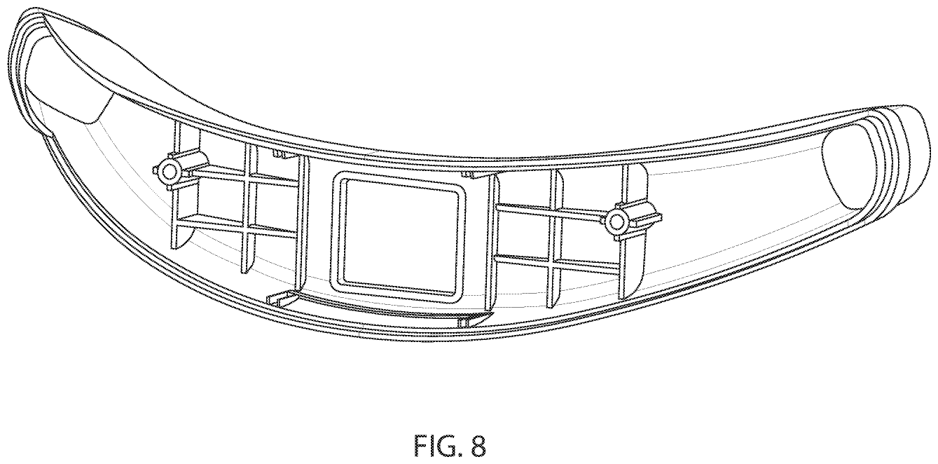

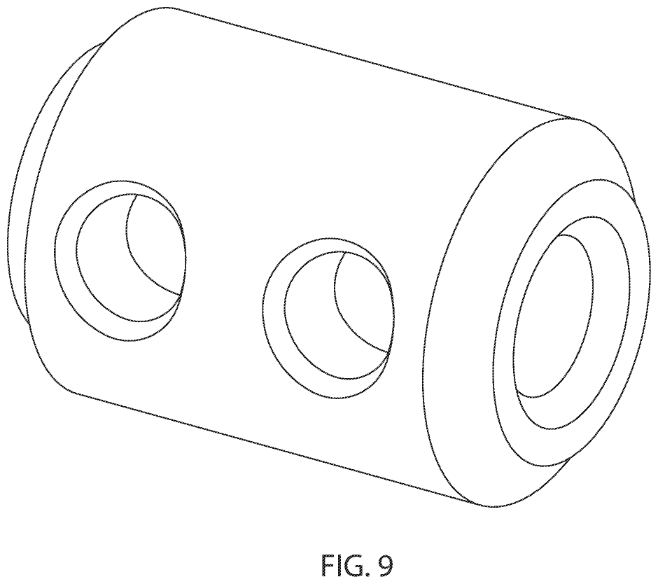

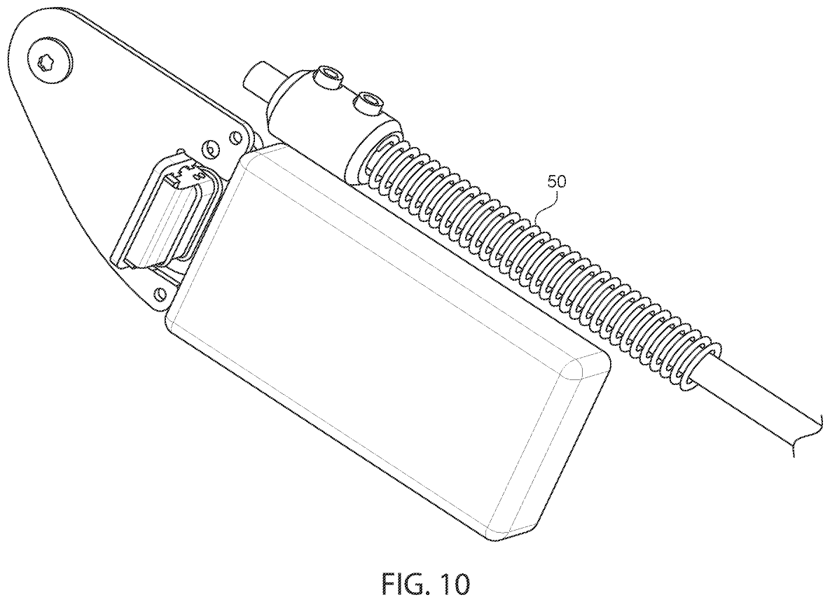

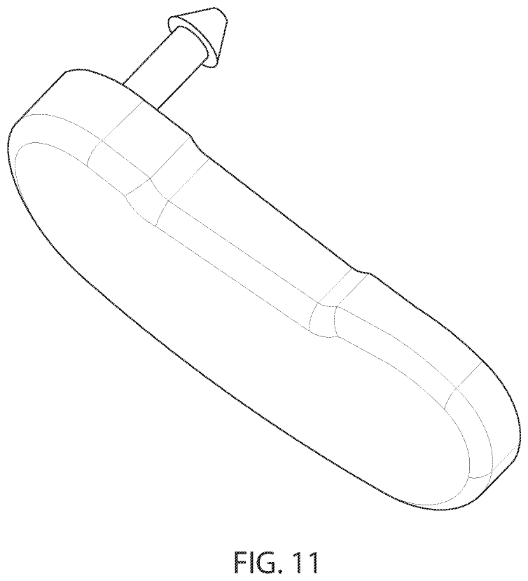

Referring to FIGS. 1 through 11, the present invention may include neck-worn medical device 100. The neck-worn medical device 100 has a main body 20 having a flexure element 30 on each opposing end. Between the flexure elements 30, along an outer surface of the main body 20, is a monitoring interface 22. Operatively associated with the monitoring interface 22, and housed in the main body 20, are one or more physiological monitor devices.

The one or more physiological monitor devices may include sensors, biomarkers, or the like, that when the monitoring interface 22 has effective skin contact, can sense physiological parameters. The one or more physiological monitor devices may include sensors, biomarkers, or the like, that when the monitoring interface 22 has effective skin contact, can sense physiological parameters continuously. The one or more physiological monitor devices may include sensors, biomarkers, or the like, that when the monitoring interface 22 has effective skin contact, can sense physiological parameters in real-time.

The one or more physiological monitor devices may include sensors, biomarkers, or the like, that when the monitoring interface 22 has effective skin contact, can sense predetermined physiological parameters. The one or more physiological monitor devices may include sensors, biomarkers, or the like, that when the monitoring interface 22 has effective skin contact, can sense predetermined physiological parameters continuously. The one or more physiological monitor devices may include sensors, biomarkers, or the like, that when the monitoring interface 22 has effective skin contact, can sense predetermined physiological parameters in real-time.

The one or more physiological monitor devices may include sensors, biomarkers, or the like, that when the monitoring interface 22 has effective skin contact, can sense or determine a disease or disorder. The one or more physiological monitor devices may include sensors, biomarkers, or the like, that when the monitoring interface 22 has effective skin contact, can sense or determine a disease or disorder continuously. The one or more physiological monitor devices may include sensors, biomarkers, or the like, that when the monitoring interface 22 has effective skin contact, can sense or determine, for example, a disease or disorder in real-time.

The one or more physiological monitor devices may include sensors, biomarkers, or the like, that when the monitoring interface 22 has effective skin contact, can sense or monitor treatment of disease or disorder. The one or more physiological monitor devices may include sensors, biomarkers, or the like, that when the monitoring interface 22 has effective skin contact, can sense or monitor treatment of disease or disorder continuously. The one or more physiological monitor devices may include sensors, biomarkers, or the like, that when the monitoring interface 22 has effective skin contact, can sense or monitor for example treatment of disease or disorder, in real-time.

Extending from each flexure element 30, away from the main body 20, is an arm portion 40. Each arm portion 40 is generally arcuate and duck-feet shaped as it extends from a smaller proximal portion 42 connected to the flexure element 30 to a wider distal portion 44. The distal portion 44 defines an inner compartment 46 for housing, among other things, a power source 60. The flexure element 30 may be made from thermoplastic elastomers or other materials with both thermoplastic and elastomeric properties, including the ability to stretch to moderate elongations and return to its near original shape allowing sufficient physical range of movement to enable the disclosure herein. The flexure element 30 enables the arm portions to move independent of the main body 20 so stretching the arm portions 40, across a plurality of actuated positions, around the neck of the wearer yet have the arm portions 40 return to their original shape, form, and biased position.

Also housed in the distal portion 44 is a biasing component 50. Each biasing component 50 operatively associates with a securement member 52 that projects from the distal portion 40 and terminates at a node 54 for electrode attachment for operatively associating an electrode to a wearer's body. The biasing component 50 urges the node 54 (and thus any patient-facing surface of an attached electrode) against the chest of the wearer selectively sot hold the sensing portion 30 in proper position for sensing electrodes attached to its rear, patient-facing surface to be attached to the proper locations on the patient's chest.

Generally speaking, such a configuration of the biasing component 50 in the distal arm portions 40, the monitoring interface 22 centrally disposed in the main body 20, and the flexure element 30 interconnecting the two serves to hold the electrode nodes 54 at their proper positions along the patient's chest, while urging effective skin contact of the monitoring interface 22 against the nape of the neck. The biasing component 50 may be a spring.

As used in this application, the term “about” or “approximately” refers to a range of values within plus or minus 10% of the specified number. By way of non-limiting example, the term “about ten (10)” would encompass nine (9) to eleven (11) or 9-11. The term “substantially” refers to up to 80% or more of an entirety. Recitation of ranges of values herein are not intended to be limiting, referring instead individually to any and all values falling within the range, unless otherwise indicated, and each separate value within such a range is incorporated into the specification as if it were individually recited herein.

For purposes of this disclosure, the term “aligned” means parallel, substantially parallel, or forming an angle of less than 35.0 degrees. For purposes of this disclosure, the term “transverse” means perpendicular, substantially perpendicular, or forming an angle between 55.0 and 125.0 degrees. Also, for purposes of this disclosure, the term “length” means the longest dimension of an object. Also, for purposes of this disclosure, the term “width” means the dimension of an object from side to side. For the purposes of this disclosure, the term “above” generally means superjacent, substantially superjacent, or higher than another object although not directly overlying the object. Further, for purposes of this disclosure, the term “mechanical communication” generally refers to components being in direct physical contact with each other or being in indirect physical contact with each other where movement of one component affect the position of the other.

Generally speaking, subjects may be exposed to different monitoring equipment. The monitoring equipment may be for example wellness/fitness type of monitoring equipment such as different smart watches or medical monitoring equipment such as ICU equipment. The boundaries between the wellness/fitness monitoring equipment and the medical monitoring equipment is becoming more and more overlapping as a result of the ever, increasing understanding and appreciation of the physiologically relevance different bodily functions and biomarkers play in both wellness/fitness and the general health status of a subject.

The device of the present invention may be used to measure important bodily functions and parameters, such as for example heart rate, blood pressure, blood volume, oxygen levels, sugar levels, blood ketone levels, purine levels, liver enzyme activity and haemoglobin levels.

In some embodiments of the present invention, the device is configured to measure or monitor heart rate, blood pressure, blood volume, oxygen levels, sugar levels, blood ketone levels, purine levels, liver enzyme activity and haemoglobin levels on a subject.

In some embodiments of the present invention, the device is configured to measure or monitor continuously, heart rate, blood pressure, purine levels, sugar levels, blood ketone levels, liver enzyme activity and haemoglobin levels on a subject.

In some embodiments of the present invention, the device is configured to measure or monitor continuously in real-time, heart rate, blood pressure, purine levels, sugar levels, blood ketone levels, liver enzyme activity and haemoglobin levels on a subject.

Subjects can also be exposed to different types of monitoring equipment to measure and monitor for example biomarkers and/or parameters such as an electrocardiogram (ECG); respiration rate (RR); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); photoplethysmography (PPG); mean corpuscular hacmoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time (PTT); hypoxanthine; and C-reactive protein (CRP).

Data collected by the device can be processed and presented as mean standard deviation (mSD).

In some embodiments of the present invention, the device is configured to measure or monitor biomarkers and/or parameters such as an electrocardiogram (ECG); respiration rate (RR); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); mean corpuscular haemoglobin concentration (MCHC); mean corpuscular haemoglobin (MCH); photoplethysmography (PPG); mean platelet volume (MPV); platelet distribution width (PDW); red cell distribution width (RDW); white blood cells (WBC); absolute neutrophil count (ANC); activated partial thromboplastin time (aPTT); partial thromboplastin time (PTT) and C-reactive protein (CRP) on a subject.

In some embodiments of the present invention, the device is configured to measure or monitor continuously biomarkers and parameters such as an electrocardiogram (ECG); respiration rate (RR); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); photoplethysmography (PPG); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time (PTT); hypoxanthine; and C-reactive protein (CRP) on a subject.

In some embodiments of the present invention, the device is configured to measure or monitor continuously in real-time biomarkers and parameters such as an electrocardiogram (ECG); respiration rate (RR); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time (PTT); hypoxanthine; and C-reactive protein (CRP) on a subject.

In some instances, sensing devices may be used for pulse oximetry, which may be an effective and quick way to monitor heart and lung function of a person. These pulse oximetry devices may be capable of evaluating the colour of blood as the amount of oxygen carried by the haemoglobin may affect the colour of blood. In some examples, a pulse oximetry device may be placed on a wearer to measure the oxygenation of the person's blood.

In some embodiments, the device of the present invention is configured to monitor, such as continuously monitor in real-time, partial pressure of oxygen PaO₂.

In some embodiments, the device of the present invention continuously monitors partial pressure of oxygen PaO₂ when in contact against skin adjacent the cervical vertebrae and sternal portions of the wearer.

In some embodiments, the device of the present invention monitors, such as continuously in real-time monitors, oxygen saturation SpO₂.

In some embodiments, the device of the present invention continuously monitors oxygen saturation SpO₂ when in contact force against skin adjacent the cervical vertebrae and sternal portions of the wearer.

Monitoring, such as continuous monitoring or continuous monitoring in real-time of cardiac rhythm, is also contemplated in the present invention which can enable transformative diagnostic and patient management tools.

In some embodiments, sensors are incorporated in the device allowing continuous monitoring of cardiac rhythm.

In some embodiments, optical sensors are incorporated in the device allowing continuous monitoring of blood volume variations referred to as photoplethysmography (PPG) from which the heart rate and other physiological parameters can be extracted to inform about user activity, fitness, sleep, and health.

In some embodiments, the device of the present invention monitors cardiac rhythm. In some embodiments, the device of the present invention monitors cardiac rhythm. In some embodiments, the device of the present invention continuously monitors cardiac rhythm.

In some embodiments, the device of the present invention monitors PPG. In some embodiments, the device of the present invention monitors PPG. In some embodiments, the device of the present invention continuously monitors PPG. In some embodiments, the device of the present invention continuously in real-time monitors PPG.

### Physiological Monitor Devices

The skilled person in the art would readily understand that different physiological monitor devices such as sensors, biomarkers, or the like can be incorporated in the present system or medical device to monitor or measure for example physiological parameters continuously or continuously in real-time on a subject.

In some embodiments, the device comprises an electrode sensor.

In some embodiments, the device comprises an optical sensor.

In some embodiments, the device comprises a light-emitting diode (LED). In some embodiments, the LED comprises a near-infrared LED.

In some embodiments, the device comprises a photodiode.

In some embodiments, the device comprises a laser.

In an embodiment, the device comprises one or more from the group consisting or an electrode sensor, an optical sensor, light-emitting diode (LED) sensor, a near-infrared LED sensor, a photodiode sensor and a laser.

The choice of the particular physiological monitor device may be influenced by different factors such as for instance the type of physiological biometer or parameter which is to be monitored or measures. Other contributory factors to the choice of physiological monitor device include proximity of the blood vessels to the skin surface around the neck of the wearer.

A particular factor which may influence the choice of the physiological monitor device is the proximity to the spinal column at the nape of the neck of the wearer. The proximity to the spinal column may facilitate more accurate measurements when detecting or monitoring physiological biomarkers or parameters such as for instance heart rate, pulse or blood oxygen level.

A further factor which may influence the choice of the physiological monitor device is the proximity cervical vertebrae and sternal portions of the wearer. The proximity to the cervical vertebrae and sternal portions of the wearer may facilitate more accurate measurements when detecting or monitoring physiological biomarkers or parameters such as for instance heart rate, pulse or blood oxygen level.

Additionally, due to the skin area of use around the neck, the physiological monitor device may be useful for controlled environments. The area around the neck of the wearer may be particularly useful for monitoring daily routine activities, during fitness training or during sleep.

In particular, due to the discretely small area of the spinal column at the nape of the neck of the wearer, the physiological monitor device would allow richer data source to be accessed namely the spinal column, leading to more accurate monitoring of physiological biomarkers or parameters and confidence in the data.

### Data Processing

In some embodiments, the data collected from the device has to be processed on the device or remotely. After normalisation and pre-processing of the data, MetIDQ™ software (Biocrates) can be used for peak integration and calculation of biomarkers. If the measurements were outside the measurable range, values were imputed as follows: concentrations below the detection limit (LOD) was set to half of the lowest measured concentrations. Concentrations below the limit of quantification (LOQ) can be set to half of the LOQ. In addition, concentration higher than the highest calibration standard concentration can be set to the highest standard concentrations.

The present invention recognises that personal information data, including the physiological biomarker data acquired using the physiological monitor device of the present invention, can be used for the benefit of the wearer and/or others.

Further, other uses for personal information data, including biometric data that benefit the user are also contemplated by the present disclosure.

The present disclosure further contemplates that the entities that may be responsible for the collection, analysis, disclosure, transfer, storage, or other use of any personal information data will comply with well-established privacy policies and/or privacy practices. In particular, such entities should implement and consistently use privacy policies and practices that are generally recognised as meeting or exceeding industry or governmental requirements for maintaining personal information data private and secure, including the use of data encryption and security methods that at least meets or may even exceed industry or government standards.

### Statistical Analysis

Analysis can be carried out on the device itself or remotely using systems and software programs known in the art such as for example IBM SPSS version 25. Variables in measurement and data with skewed distributions can be log-transformed to ensure normality. Comparisons can be performed with t-test, Wilcoxon-Mann-Whitney, and one-way ANOVA as appropriate. Significance was defined as p<0.05. Non-parametric tests were used for comparing ordinal or non-normal variables. Data can be presented as mean standard deviation (mSD).

### Definitions

As used herein, the term “biomarker” refers to a physiological characteristic or physiological parameter that can be objectively measured by for example known sensors and evaluated for example by using known statistical methods, as an indicator of normal and/or disease processes, pharmacological responses or physiological status of a subject. A “biomarker” can be used to measure the onset or the progress of a disease, the effects of treatment or regimen, or provide information on user activity, fitness, sleep, health and metabolic status. One of the advantages of the medical device described herein is that biomarker measurements can be collected without disruption of the skin or direct contact with the blood supply of the wearer.

In some embodiment, the biomarker is a predetermined physiological parameter.

In some embodiments, the parameter can be chemical, physical or biological.

The parameter can be altered or modified where the alteration can be measured or evaluated continuously or continuously in real-time by the device in situ i.e. while in place on a subject.

The parameter can be altered or modified where the alteration can be measured or evaluated continuously or continuously in real-time by the device remotely.

In some embodiments, the biomarker or parameter can be selected from one or more of the group consisting of electrocardiogram (ECG); respiration rate (RR); cardiac rhythm (CR), photoplethysmography (PPG); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time (PTT); hypoxanthine; and C-reactive protein (CRP).

As used herein, the term “alteration” may be used interchangeably with the terms, “alter” or “modify” such as increase or decrease in the level of a metabolite such as a chemical or a biomarker detected and/or analysed and/or monitored, by the device of the present invention.

In some embodiments, the alteration is at least 0.01%, 0.05%, 0.1%, 0.15%, 0.2%, 0.4%, 1%, 2%, 2.5%, 5%, 10%, 15%, 20%, 25%, 30%, 35%, 40%, 45%, 50%, 55%, 60%, 65%, 70%, 75%, 80%, 85%, 90%, 95% or 100% or greater compared to control or base level.

In some embodiments the alteration may be at least 0.1, 0.25, 0.5, 1, 1.5, 2, 2.5, 3, 3.5, 4, 4.5, 5, 5.5, 6, 6.5, 7, 7.5, 8, 8.5, 9, 9.5, or 10-fold or greater compared to control or base level.

In some embodiments the alteration of the parameter is statistically significant. In some embodiments the effectiveness or the device of the present invention is determined qualitatively.

In some embodiments the effectiveness of the device of the present invention is determined quantitatively.

In some embodiments the alteration is determined qualitatively. In some embodiments the alteration is determined quantitatively.

In some embodiments, alteration is assessed by a qualitative step and/or a quantifying step. In further embodiments, the qualitative step and/or a quantifying step is performed on a sample using the device of the present invention.

In further embodiments, the qualitative step and/or a quantifying step is performed on a subject using the device of the present invention.

In further embodiments, the qualitative step and/or a quantifying step is performed continuously.

In further embodiments, the qualitative step and/or a quantifying step is performed in real-time. In further embodiments, the qualitative step and/or a quantifying step is performed continuously in real-time.

As used herein, “treating” or “treatment” of any disease or disorder refers, in certain embodiments, to ameliorating a disease or disorder that exists in a subject. “Treating” or “treatment” includes ameliorating at least one physiological or physical parameter, which may be indiscernible by the subject. In yet another embodiment, “treating” or “treatment” includes modulating the disease or disorder, either physically (e.g., stabilization of a discernible symptom) or physiologically (e.g., stabilisation of a physical parameter) or both.

In yet another embodiment, “treating” or “treatment” includes delaying or preventing the onset of the disease or disorder.

Further, as used herein, “treatment” includes preventing or delaying the recurrence of the disease, delaying or slowing the progression of the disease, ameliorating the disease state, providing a remission (partial or total) of the disease, decreasing the dose of one or more other medications required to treat the disease, delaying the progression of the disease, increasing or improving the quality of life, increasing weight gain, and/or prolonging survival. Also encompassed by “treatment” is a reduction of pathological consequence of a disease e.g., metabolic condition, disorder or severe illness.

As used herein the terms “subject” and “wearer” are used interchangeably. As used herein, the term “subject” means any animal, such as a vertebrate, preferably a mammal such as human. Preferably the subject wears or has been fitted with the medical device of the present invention.

The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” “top,” “bottom,” “up,” “down”, “upper”, “lower”, “above”, “below”, “beneath”, “front”, “back”, “over”, “under”, “left”, “right”, etc. are used with reference to the orientation of some of the components of the medical device of the present invention. Since constituents or components in various embodiments described here can be positioned in a number of different orientations, directional terminology is used for purposes of illustration only and is in no way limiting. The directional terminology is intended to be construed broadly, and therefore should not be interpreted to preclude components being oriented in different ways.

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. It will also be appreciated that the device(s), method(s), use(s), detector(s), sensor(s), physiological biomarker(s) may be subject to numerous rearrangements, modifications and substitutions without departing from the scope of the present disclosure as set forth and defined by the following claims.

What is claimed is:

1. A neck-worn medical device, comprising:
a monitoring interface having a main housing, the main housing terminating in opposing flexure elements;
an arm portion operatively associated with each flexure element so that each arm portion is movable, relative the main housing, between a biased position and a plurality of actuated positions;
a distal end of the arm portion houses a biasing component; and
a securement member projecting from the biasing component until it terminates at a node for a chest sensor, wherein the biasing component is configured, with the arm portions in the biased position, to urge the node against a sternal portion of a wearer and urge a nominal contact force between the monitoring interface and a nape portion of the wearer.

2. A neck-worn medical device according to claim 1, wherein operatively associated with the monitoring interface, and housed in the main body, are one or more physiological monitor devices.

3. A neck-worn medical device according to claim 2, wherein the one or more physiological monitor devices include sensors, biomarkers, or the like, that when the monitoring interface has effective skin contact, the one or more physiological monitor devices sense or monitor physiological parameters.

4. A neck-worn medical device according to claim 3, wherein the one or more physiological monitor devices include sensors, biomarkers, or the like, that when the monitoring interface has effective skin contact, the one or more physiological monitor devices sense or monitor predetermined physiological parameters.

5. A neck-worn medical device according to claim 4, wherein the one or more physiological monitor devices include sensors, biomarkers, or the like, that when the monitoring interface has effective skin contact, the one or more physiological monitor devices sense, monitor or determine a disease or disorder.

6. A neck-worn medical device according to claim 5, wherein the one or more physiological monitor devices include sensors, biomarkers, or the like, that when the monitoring interface has effective skin contact, the one or more physiological monitor devices sense, monitor or determine treatment of disease or disorder.

7. A neck-worn medical device according to claim 6, wherein the one or more physiological monitor devices include sensors, biomarkers, or the like, wherein the device sense, monitor or determine physiological parameters, treatment, disease or disorder continuously and/or in real-time.

8. A neck-worn medical device according to claim 6, wherein the one or more physiological monitor devices include sensors, biomarkers, or the like, wherein the device sense, monitor or determine user activity, fitness, sleep, and health.

9. A neck-worn medical device according to claim 2, wherein the biomarker is one or more of the group consisting of electrocardiogram (ECG); respiration rate (RR); cardiac rhythm (CR), photoplethysmography (PPG); body mass index (BMI); alkaline phosphatase (ALP); alanine transaminase (ALT); aspartate aminotransferase (AST); arterial pH (Art pH); partial pressure of oxygen (PaO₂); oxygen saturation (SpO₂%); partial pressure of carbon dioxide (PaCO₂); red blood cell count (RBC); mean corpuscular haemoglobin concentration (MCHC); mean corpuscular haemoglobin (MCH); mean platelet volume (MPV); platelet distribution width (PDW); red cell distribution width (RDW); white blood cells (WBC); absolute neutrophil count (ANC); activated partial thromboplastin time (aPTT); partial thromboplastin time (PTT); hypoxanthine; and C-reactive protein (CRP).

10. A neck-worn medical device according to claim 2, wherein the one or more physiological monitor devices includes a biomarker comprising partial pressure of oxygen PaO₂.

11. A neck-worn medical device according to claim 2, wherein the one or more physiological monitor devices includes a biomarker comprising oxygen saturation SpO₂.

12. A neck-worn medical device according to claim 2, wherein the one or more physiological monitor devices includes a biomarker comprising cardiac rhythm.

13. A neck-worn medical device according to claim 2, wherein the one or more physiological monitor devices includes a biomarker comprising photoplethysmography (PPG).

14. A neck-worn medical device according to claim 2, wherein a subject is monitored continuously.

15. A neck-worn medical device according to claim 2, wherein a subject is monitored continuously in real-time.

16. A neck-worn medical device of claim 1, wherein each flexure element comprises thermoplastic elastomers having a tapering tubular form, wherein a first end of the tubular form engages an outer shell of the main body, and wherein a tapered second end engages an outer shell of a respective arm portion.

17. A neck-worn medical device of claim 16, wherein the first and second ends of the flexure element applies encircling pressure to said outer shells of the main body and the arm portion, respectively.

18. A neck-worn medical device of claim 17, wherein each outer shell comprises two complementary components that are held together, in part, by said encircling pressure.

19. A neck-worn medical device of claim 1, wherein each arm portion is arcuate and has a duck-feet shaped distal portion, wherein the distal portion houses a power source.

20. A neck-worn medical device of claim 1, wherein the biasing component is a spring.
